(19) United States
(12) Patent Application Publication
(10) Pub. No.: US 2018/0315343 A1
(43) Pub. Date: Nov. 1, 2018

(54) SYSTEM AND METHOD FOR TRANSLATING SOUND TO TACTILE

(71) Applicant: ALTO S2V LTD, MAALE GAMLA (IL)

(72) Inventors: SHAIE SHVARTZBERG, MAALE GAMLA (IL); URI ROM, MAALE GAMLA (IL); MOR SHVARTZBERG, MAALE GAMLA (IL)

(21) Appl. No.: 15/768,023

(22) PCT Filed: Oct. 13, 2016
(86) PCT No.: PCT/IL16/51107

§ 371 Date: Apr. 13, 2018

(30) Foreign Application Priority Data
Oct. 15, 2015 (IL) 242106

(51) Int. Cl. G09B 21/00; G10L 21/06

(52) U.S. Cl. CPC G09B 21/009; G10L 2021/065; G10L 21/06

(57) ABSTRACT

A Sound translating system configured to touch human skin comprising: a sound converter configured to process sound waves to a plurality of sound bands, wherein each sound band of the plurality of sound bands is represented by at least one electrical signal having specific electrical characteristics, wherein the sound converter is further configured to transmit the at least one electrical signal; at least one device comprising a plurality of actuators, wherein at least one actuator of the plurality of actuators is configured to convert the at least one electrical signal to mechanical vibration, wherein the mechanical vibration of each actuator stimulate tactile of one of a plurality of zones on the human skin, wherein the at least one actuator is dedicated to at least one predefined zone of the plurality of zones; and wherein, the predefined zone complies with a map used to allocate sound bands to zones on the human skin.

### TECHNICAL FIELD

The present disclosed subject matter relates to manipulating sound waves. More particularly, the present disclosure relates to systems and methods for translating sound waves into analogous tactile stimulation.

### BACKGROUND

Millions of people worldwide suffer from hearing loss. The hearing loss may be mild, moderate, severe or profound. It can affect one ear or both ears, and leads to difficulty in hearing conversational speech or loud sounds. Hearing loss may result from genetic causes, complications at birth, certain infectious diseases, chronic ear infections, use of particular drugs, exposure to excessive noise, and also due to ageing. Disabling hearing loss refers to hearing loss greater than 40 decibels (dB) in the better hearing ear in adults, and a hearing loss greater than 30 dB in the better hearing ear in children.

People suffering from hearing loss, often use sign language as means of communication but they live in a silent world. Various technical solutions have been used to help deaf and hearing impaired people to experience the “sound” world. For example, there are dedicated dance stages in clubs of the “Association of the Deaf”, whereby these stages are modified with large speakers that transfer the sound-waves to the floor so as to be sensed by the deaf dancers in the club.

Additionally, some deaf people hold household balloons (filled with air), that are affected by the sound waves in the air and therefore help experiencing some sounds, for instance gunshots or drums in a movie. However, when a pen moves on the table, a door squeak or talking occurs, deaf people cannot feel any change and the sound experience is therefore not complete.

Some commercially available solutions have tactile abilities whereby a sound triggers a vibration. Other commercially available solutions have wearable devices that translate a sound to a vibration. Thus, a user can recognize that there is music playing but cannot understand what kind of sound it is.

However, none of the solutions provide a system that can discriminate sound waves (and particularly frequencies and harmonics) such that the individual components of any sound wave are translated differently into vibrations. It is therefore an object of the present disclosed subject matter to provide systems and methods of modifying sound waves with corresponding tactile feedback, such that each sound component is analyzed individually. Further objects and advantages of this subject matter will be disclosed in the detailed description.

### SUMMARY

One primary aspect of the present disclosure is a Sound translating system configured to touch human skin comprising: a sound converter configured to process sound waves to a plurality of sound bands, wherein each sound band of the plurality of sound bands is represented by at least one electrical signal having specific electrical characteristics, wherein the sound converter is further configured to transmit the at least one electrical signal; at least one device comprising a plurality of actuators, wherein at least one actuator of the plurality of actuators is configured to convert the at least one electrical signal to mechanical vibration, wherein the mechanical vibration of each actuator stimulate tactile of one of a plurality of zones on the human skin, wherein the at least one actuator is dedicated to at least one predefined zone of the plurality of zones; and wherein, the predefined zone complies with a map used to allocate sound bands to zones on the human skin.

In some exemplary embodiments, the at least one actuator is further configured to modulate an amplitude and a frequency of the mechanical vibration by the specific electrical characteristics of the at least one electrical signal.

In some exemplary embodiments, the sound converter is selected from the group consisting of: a smart phone; notebook computer; tablet; and a desktop computer utilized to process applications for implementing sound converter functionalities.

In some exemplary embodiments, the sound converter is hosted in a dedicated base unit comprising a Digital Signal Processor (DSP) programmed for implementing sound converter functionalities.

In some exemplary embodiments, the sound converter receives the sound waves by a microphone and wherein, the sound converter is further configured to process sound signals selected from the group consisting of: Bluetooth signals; AM signals; FM signals; digital audio signals and analogue audio signals.

In some exemplary embodiments, the sound band is selected from the group consisting of: a single frequency; harmonic series; a band of adjacent frequencies, and wherein the specific electrical characteristics of the electrical signal are indicative of at least one sound band; and wherein the electrical signal is wirelessly transmitted to the at least one device.

In some exemplary embodiments, the device is selected from the group consisting of: a bodysuit; gloves; mat and handball comprise a plurality of actuators, wherein each actuator of the plurality of actuators is allocated to overlap a predetermined zone on the human skin, and, wherein each actuator is associated with one or more sound bands selected from the group consisting of: tones; acoustic harmonies; frequency ranges and acoustic instruments.

In some exemplary embodiments, the sound converter is further configured for assembling a plurality of the at least one electrical signal into a primary signal and transmit the primary signal to the device.

In some exemplary embodiments, the device further comprising a driver configured to receive a primary, wherein the driver is further configured to split the primary signal to a plurality of the at least one electrical signal; and wherein the at least one electrical signal is connected to at least one actuator of the plurality of actuators, and wherein the device and the driver are physically separated.

Another primary aspect of the present disclosure is a sound translating device configured to touch a human skin comprising: a sound converting component configured to process sound waves to a plurality of sound bands, wherein each sound band of the plurality of sound bands is represented by at least one electrical signal having specific electrical characteristics, a plurality of actuators, wherein at least one actuator of the plurality of actuators is configured to convert the at least one electrical signal to mechanical vibration, wherein the mechanical vibration of each actuator stimulate tactile of one of a plurality of zones on the human skin, wherein the at least one actuator is dedicated to at least one predefined zone of the plurality of zones; and wherein, the predefined zone complies with a map used to allocate sound bands to zones on the human skin.

In some exemplary embodiments, the at least one actuator is further configured to modulate an amplitude and a frequency of the mechanical vibration by the specific electrical characteristics of the at least one electrical signal.

In some exemplary embodiments, the sound converting component utilizes a Digital Signal Processor (DSP) programmed for implementing sound converting functionalities.

In some exemplary embodiments, the sound converting component receives the sound waves by a microphone and wherein, the sound converting component is further configured to process sound signals selected from the group consisting of: Bluetooth signals; AM signals; FM signals; digital audio signals and analogue audio signals.

In some exemplary embodiments, the sound band is selected from the group consisting of: a single frequency; harmonic series; a band of adjacent frequencies, and In some exemplary embodiments, the specific electrical characteristics of the electrical signal are indicative of at least one sound band.

In some exemplary embodiments, the device is selected from the group consisting of: a bodysuit; gloves; mat and handball comprise a plurality of actuators, wherein each actuator of the plurality of actuators is allocated to overlap a predetermined zone on the human skin, and wherein each actuator is associated with one or more sound bands selected from the group consisting of: tones; acoustic harmonies; frequency ranges and acoustic instruments.

In some exemplary embodiments, the sound converter component is further configured for assembling a plurality of the at least one electrical signal into a primary signal and transmit the primary signal to the device.

In some exemplary embodiments, wherein said device further comprising a driver configured to receive a primary, wherein the driver is further configured to disband the primary signal to a plurality of the at least one electrical signal; and wherein the at least one electrical signal is connected to at least one actuator of the plurality of actuators, and wherein said device and said driver are physically separated.

Yet another primary aspect of the present disclosure is a method for translating sound into mechanical vibration on human skin, the method comprising: receiving sound signals selected from the group consisting of: microphone signals; Bluetooth signals; AM signals; FM signals; digital audio signals and analogue audio signals; continuously analyzing the sound signals amplitude and frequencies; continuously determining a plurality of sound bands to reflect content of the sound based on the sound signals amplitude and frequencies; continuously converting each sound band of the plurality of sound bands to an electrical signal of a plurality of electrical signals, wherein each electrical signal of the plurality of electrical signals have a specific electrical characteristics that represent the sound band; transmitting the plurality of electrical signals to a device comprising a plurality of actuators, wherein each actuator of the plurality of actuators is activated by a matching electrical signal of the plurality of electrical signals; wherein each actuator transform the energy of the matching electrical signal to a mechanical vibration; and wherein the mechanical vibration stimulate tactile of one of a plurality of zones on the human skin.

In some exemplary embodiments, wherein said converting further comprises modulating an amplitude and a frequency of an electrical signal to satisfy mechanical vibration characteristics of an actuator.

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present disclosed subject matter, suitable methods and materials are described below. In case of conflict, the specification, including definitions, will control. In addition, the materials, methods, and examples are illustrative only and not intended to be limiting.

### BRIEF DESCRIPTION OF THE DRAWINGS

The disclosed subject matter is herein described, by way of example only, with reference to the accompanying drawings. With specific reference now to the drawings in detail, it is stressed that the particulars shown are by way of example and for purposes of illustrative discussion of the preferred embodiments of the present disclosed subject matter only, and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the subject matter, the description taken with the drawings making apparent to those skilled in the art how the several forms of the subject matter may be embodied in practice.

In the drawings:

FIG. 1 shows a block diagram of a system for translating sound to tactile, in accordance with some exemplary embodiments of the disclosed subject matter;

FIG. 2A illustrates a sound to tactile system with on-hook conch devices, in accordance with some exemplary embodiments of the disclosed subject matter;

FIG. 2B illustrates a sound to tactile system with off-hook conch devices, in accordance with some exemplary embodiments of the disclosed subject matter;

FIG. 3A illustrates a cross-sectional view of a conch device, in accordance with some exemplary embodiments of the disclosed subject matter;

FIG. 3B illustrates several configurations of holding a conch device, in accordance with some exemplary embodiments of the disclosed subject matter;

FIG. 4 illustrates a glove comprising a plurality of actuators and a driver, in accordance with some exemplary embodiments of the disclosed subject matter; and

FIG. 5 shows a flowchart diagram of a method for translating sound to tactile, in accordance with some exemplary embodiments of the disclosed subject matter.

### DETAILED DESCRIPTION

The disclosed subject matter is described below with reference to flowchart illustrations and/or block diagrams of methods, apparatus (systems) and computer program products according to embodiments of the subject matter. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

Before explaining at least one embodiment of the subject matter in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

One of the objective dealt with by the disclosed subject matter is to provide hearing impaired people with a novel personal tool that will assist them experience detailed hearing indirectly however intuitive. That is to say communicating the filling of sounds to hearing impaired people in substantially enhanced way. Some commercially available devices transform sound yet indiscriminate of frequency, harmonics, sensitive areas and tactile sensing. With such commercially available devices, a hearing impaired person may recognize that there is sound however may not be able to distinguish between the type, the amount and the magnitude of sounds.

The disclosed technical solution utilizes digital signal processing algorithm on any obtained audio signal in order to analyze and decompose the signal to its most significant sound components. This plurality of sound components continually represents the human hearing spectrum of the sound signal. In some exemplary embodiments, a sound component may represent, for example, alarm sound, baby cry, motorcycle noise, singing, shotgun sound, humming bird, dog barking, a specific musical instrument, animal sounds, male voice, female voice, children voice, vehicle noise, a combination thereof, or the like. In some exemplary embodiments of the present disclosure each sound component of the plurality of sound components may be allocated to one dedicated actuator of plurality actuators. Wherein, each actuator is adapted to transform a sound component representation to a mechanical vibration.

In some exemplary embodiments, the plurality of actuators may be incorporated (assembled) in a device such as a glove; namely, each actuator may be touching a different zone of the hand skin. The outcome of the system described above may be that each sound component is mapped to a specific zone on the skin and can stimulate tactile of that zone of the skin. Thus, after some training, a hearing impaired (or not) person may be able to differentiate between sound effects and their magnitude.

One technical effect of utilizing the disclosed subject matter is that it provides hearing impaired people a new opportunity to experience, feel, understand and interpret music and sounds.

Another technical effect of utilizing the disclosed subject matter is to provide hearing impaired people with the ability to enjoy various music players, to be part of the audience in a movie theatre or concert hall, and to participate in the social audio world.

Yet another technical effect of utilizing the disclosed subject matter is that after practice, one can interpret the vibrations and understand not just that there is music, but what kind of sound it is. Users of the present disclosure “translating sound to tactile system” can tactilely feel with their fingers or other parts of their skin the vibrations according to the audio signals', namely a kind of braille for the hearing impaired.

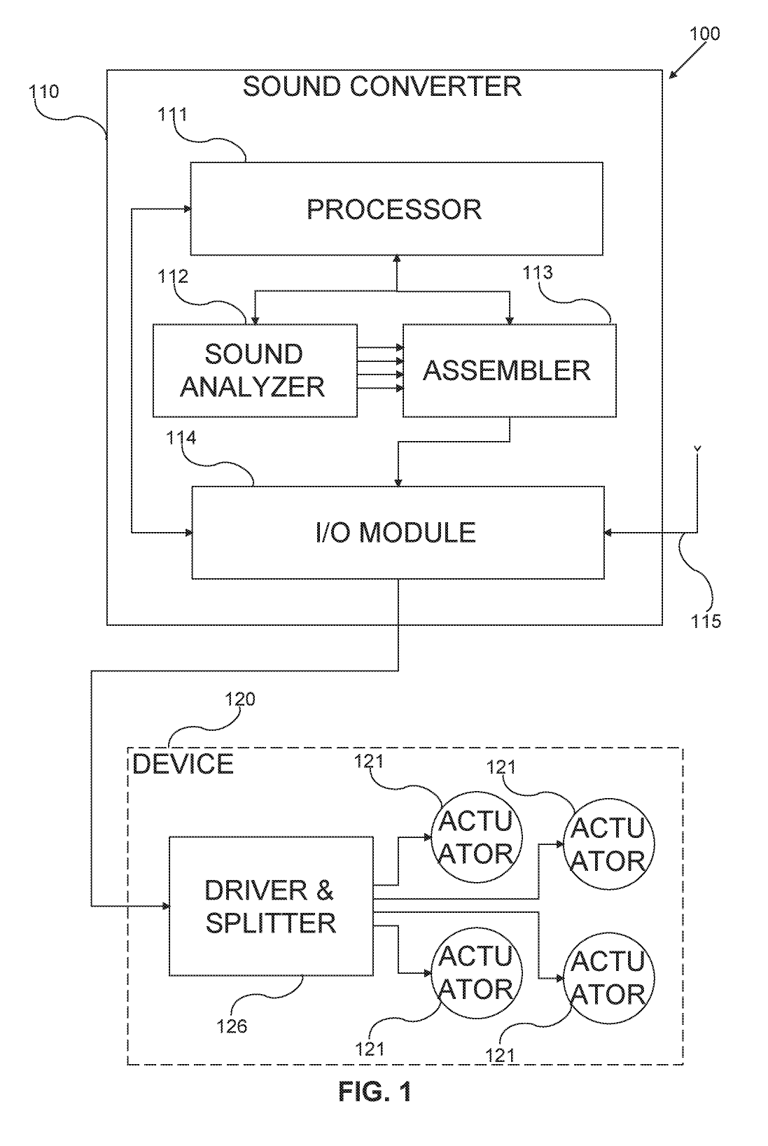

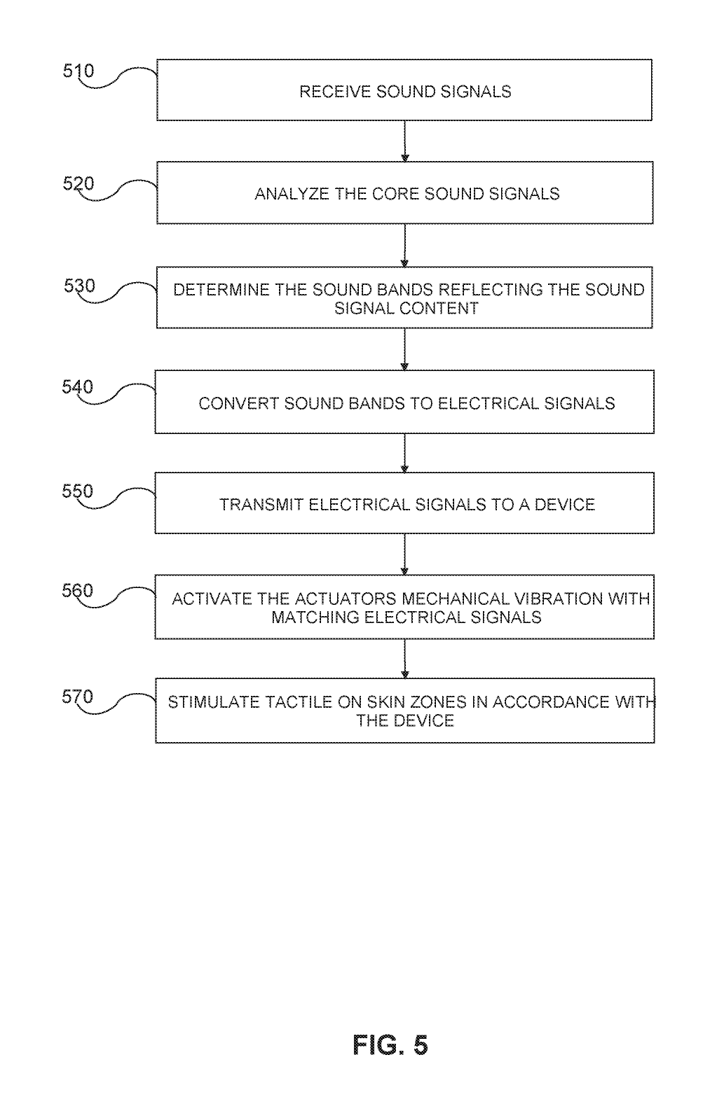

Referring now to FIG. 1 illustrating a block diagram of a Sound to Tactile Translating System (STTS) 100, in accordance with some exemplary embodiments of the disclosed subject matter. STTS 100 may be a computerized system adapted to perform the methods depicted in FIG. 5.

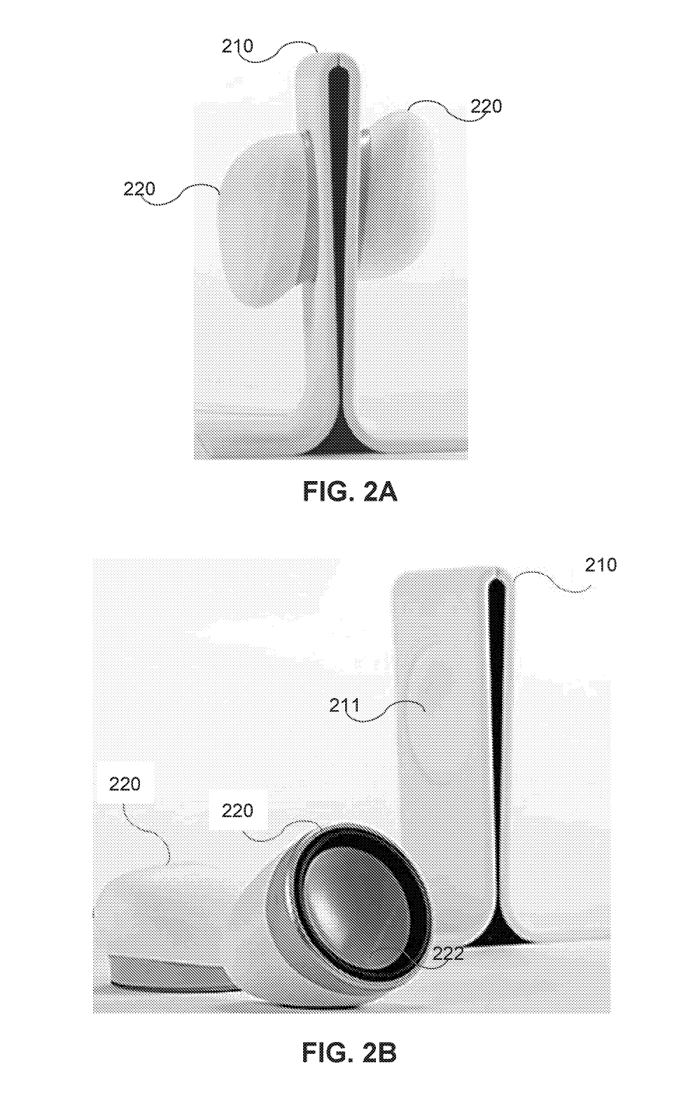

In some exemplary embodiments, STTS 100 may comprise a Sound Converter 110. Sound Converter 110 may comprise capabilities of: sound transceiver; sound analyzer; sound synthesizer; signal processing unit; a combination thereof, or the like. In some exemplary embodiments, Sound Converter 110 may be housed within a Docking Base 210 depicted in FIGS. 2A and 2B. Sound Converter 110 may comprise a Processor 111. Processor 111 may be a Central Processing Unit (CPU), a microprocessor, an electronic circuit, an Integrated Circuit (IC) or the like. Additionally or alternatively, Processor 111 can be implemented as firmware written for or ported to a specific processor such as Digital Signal Processor (DSP) or microcontrollers, or can be implemented as hardware or configurable hardware such as Field Programmable Gate Array (FPGA) or Application Specific Integrated Circuit (ASIC). Processor 111 may be utilized to perform computations required by STTS 100 or any of it subcomponents.

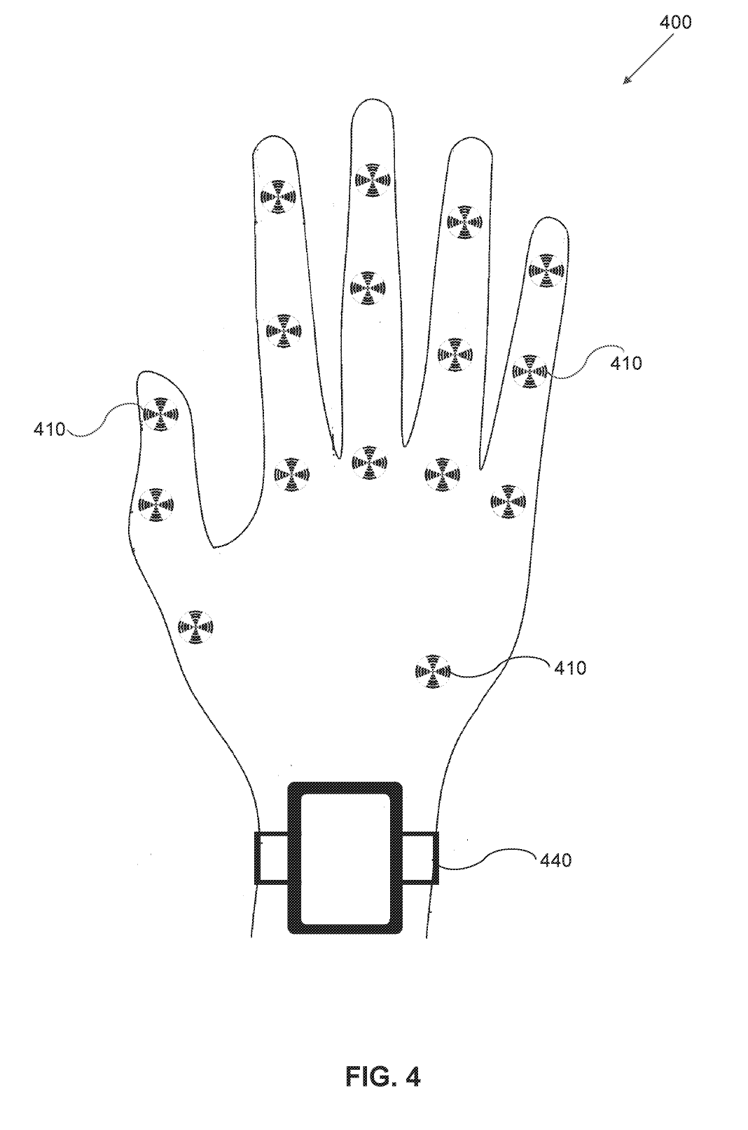

In some exemplary embodiments of the disclosed subject matter, Sound Converter 110 may comprise an Input/Output (I/O) Module 114. The I/O Module 114 may be utilizes as an interface to transmit and/or receive information and instructions between Sound Converter 110 and devices, such as a Conch device 220 of FIGS. 2A & 2B, Glove 400 of FIG. 4, or the like. Additionally or alternatively I/O Module 114 may be equipped with Antenna 115 for receiving sound signals from a variety of wireless technologies, such as Wi-Fi, Bluetooth; AM radio; FM radio; digital audio broadcast, a combination thereof, or the like. The Sound Converter may also be capable to receive audio signal over weird media from instruments, such as a microphone, a media player, amplifier, smartphone, or the like. In some exemplary embodiments, the I/O module may process signals arriving from a variety of physical media technologies in order to extract a core sound signal that represents relevant sound waves. In addition, the I/O module may also shape the gain and filter out noises from the core sound signal.

In some exemplary embodiments, an internet connection (not shown) may be used to connect STTS 100, the Internet connection may facilitate processes of communicating to a cloud base data repository that may comprise information associated with sound patterns representing, for example, musical instruments, animal sounds, male voice, female voice, children voice, vehicle noise, a combination thereof, or the like.

In some exemplary embodiments, Sound Converter 110 may comprise a Memory module (not shown). The Memory module may be persistent or volatile, for example, the memory module can be a flash disk, a random access memory, a memory chip, an optical storage device such as a CD, a DVD, or a laser disk; a magnetic storage device such as a tape, a hard disk, a semiconductor storage device such as flash device, memory stick, a combination thereof, or the like. In some exemplary embodiments, the memory module may retain program code to activate Processor 111 to perform acts associated with steps shown in FIG. 5. The memory module may also be used to retain sound patterns representation, look-up-tables mapping sound bands to zones (further described hereinafter), or the like.

In some exemplary embodiments, Sound Converter 110 may comprise a Sound Analyzer 112. Sound Analyzer 112 may be based on Digital Signal Processor (DSP) programmed for implementing sound analyzing and sound converting functionalities. In some exemplary embodiments, the sound analyzer may receive core sound signals from I/O module 114, which were originally received by a microphone, a Bluetooth signal, an ear jack, Wi-Fi signal, optical cord, or the like. By continuously analyzing the amplitude and frequencies of the core sound signal, the sound analyzer may determine properties of sound bands that make up the core sound signal. In some exemplary embodiments, the sound bands properties may be characterized by a single frequency, harmonic series, amplitude thresholds, band of adjacent frequencies, a combination thereof, or the like. Each sound band of a plurality of sound bands that make up the core sound signal may reflect a different acoustic effect, such as sound of: drums, voice, baby cry, motorcycle, singing, humming bird dog barking, a combination thereof, or the like. Additionally or alternatively, the sound analyzer may be configured to convert the plurality of sound bands into a plurality of electrical signals, each having specific electrical characteristics. It should be noted that the specific electrical characteristics of each electrical signal indicates a corresponding sound band. In some exemplary embodiments, converting a sound band into electrical signals may be done by modulating the amplitude and frequency of the electrical signal to match acoustic effects defined in a look-up-table. The look-up-table, stored in a data repository, may comprise information associated with sound patterns representing, for example, musical instruments, animal sounds, male voice, female voice, children voice, vehicle noise, a combination thereof, or the like.

In some exemplary embodiments, Sound Converter 110 may comprise Assembler 113. Assembler 113 may be an electronic circuit designed to aggregate the electrical signal into at least one primary signal and transmit the at least one primary signal to Device 120 (described later). The aggregation of the electrical signals produced by the sound analyzer may be done by a multiplexer, a mixer, sum amplifier, a combination thereof, or the like. In some exemplary embodiments, Assembler 113 may amplify the at least one primary signal and utilize I/O module 114 for transmitting the at least one primary signal to a device by either wires or wireless technologies, such as Bluetooth, Wi-Fi, a combination thereof, or the like.

In some exemplary embodiments, a hardware computing devices such as, for example, a smartphone, a notebook and a tablet may be utilized as a hardware platform for processing applications configured for implementing the sound converter functionalities.

In some exemplary embodiments, STTS 100 may comprise a device, such as Device 120. Device 120 may be adapted to touch a particular surface of a human skin, such as for example Conch 220 illustrated in FIG. 2B and Glove 400 illustrated in FIG. 4. The device 120 may comprise a plurality of actuators, such as Actuator 121, each of which may be dedicated to a predetermine zone of the particular surface that the device touches the skin. As an example, Glove 400 illustrated in FIG. 4 may be touching a backside surface of a hand with 16 actuators, where each actuator is allocated to a predetermine zone of the hand, as depicted in FIG. 4.

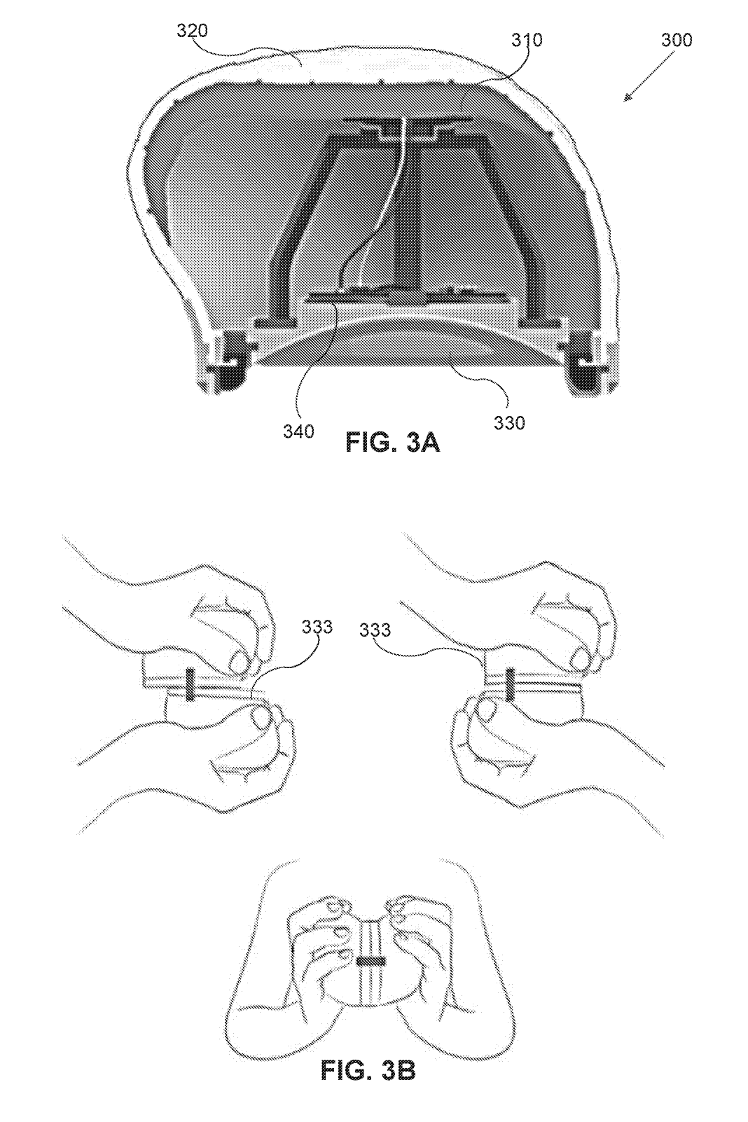

In some exemplary embodiments, each actuator, such as Actuator 121, may be driven by a dedicated electrical signal that is controlled by a driver. The actuator may be an electrical component configured to transform energy emitted by the electrical signal to mechanical vibration as a function of the amplitude and the frequency of the electrical signal. That is to say that the mechanical vibration stroke is related to the amplitude of the electrical signal and that the mechanical vibration frequency is related to the frequency electrical signal. In some exemplary embodiments, an actuator, such as Actuator 121, Actuator 310 shown in FIG. 3A and Actuator 410 shown in FIG. 4 may be a piezoelectric actuator, a linear resonant actuator, a non-linear resonant actuator, or the like.

In some exemplary embodiments, the device may comprise a driver, such as Driver & Splitter (D&S) 126. D&S 126 may be an electronic circuit designed to receive from the sound converter signals based on communication technologies, such as for example Bluetooth, Wi-Fi, or the like. In some exemplary embodiments D&S 126 may be configured extract the primary signal (i.e. represents the aggregated electrical signal) from the received signal. In some exemplary embodiments, D&S 126 may comprise a de-multiplexer, a decoder, a multi-pole filter, or the like for splitting the at least one primary signal to its components, that is, the electrical signals comprising the aggregated electrical signal. Additionally or alternatively D&S 126 may amplify the electrical signals and route each electrical signal to its associated actuator. In some exemplary embodiments, a driver, such as D&S 126, may be assembled on Printed Circuit Board (PCB), FPGA, ASIC, PLA and physically integrated within a device, such as Driver 340 shown in FIG. 3A. In some embodiments, a driver, such as Driver 440 shown in FIG. 4, may be separated from the device, such as Glove 400 shown in FIG. 4.

In some exemplary embodiments of the disclosed subject matter, Sound Converter 110 and Device 120 may be incorporated into one physical device. In such embodiments Assembler 113, a portion of I/O module 114, and a portion of D&S 126 may become redundant since the electrical signals may be wired directly to D&S 126 for amplifying and routing only.

Referring now to FIGS. 2A-2B, these figures show a Sound to Tactile Translating System (STTS) 200. FIG. 2A illustrates the STTS 200 in an on-hook state, and FIG. 2B illustrates the STTS 200 in an off-hook state. The STTS 200 comprises a Docking Base 210 and at least one detachable Conch 220. The Docking Base 210 may be powered by AC or alternatively via internal batteries. Conch 220 may be attached to and/or detached from a matching Docking Area 211 of the Docking Base 210. Preferably, the coupling between the detachable Conch 220 and the Docking Base 210 may be magnetic with corresponding magnetic elements, so as to allow easy attachment and detachment of Conch 220 from the Docking Base 210. In some embodiments, Conch 220 may comprise Battery 222 that may wirelessly receive electrical power (e.g. charged) from the Docking Base 210 using a compatible circuit for wireless power transmission.

The STTS 200 may process sounds and translate sound waves into frequency-adjusted tactile vibrations, by which hearing impaired people may “feel” the sounds by physically experiencing a tactile feedback. With such feedback, after calibration and some practice, the user may interpret the tactile vibrations into individual sound patterns (e.g. musical instruments) and thus understand not just that there is for example general music playing, but also what kind of sound it is. In this way, a new “language” may be provided for the hearing impaired community, similarly to the Braille language for the blind. With this new language for the hearing impaired, an opportunity occurs for connection to the world of music, enjoyment of a deeper experience while watching films, and also being involved in multi-participants discussions, without feeling cut off as the user may differentiate between different people talking.

In some exemplary embodiments, the functionalities of the sound converter, typically housed in Docking Base 210, may be carried out at an external computerized device, such as a smartphone, a notebook, a tablet, or the like. Thus, the external computerized device may be adapted to process applications configured for sensing, analyzing, and processing functionalities of the sound converter. In such embodiments, where an external computerized device replaces the sound converter, Docking Base 210 may be utilized as docking base as well as for housing a power supply for charging at least one Conch 220.

Referring now to FIG. 3A illustrates a cross-sectional view of a Conch 300. Conch 300 is ergonomically designed for a human hand and is covered by Layer 320 that corresponds to zones of the human hand. In some exemplary embodiments, vibrations generated by actuators such as Actuator 310, may traverse Layer 320 to corresponding zones of the human hand. In some exemplary embodiments, Conch 300 may comprise Driver 340. Driver 340 performs operations identical to the operations described for D&S 126, as shown in FIG. 1 and described hereinabove. The electrical signal output of Driver 340 may be wired directly to actuators such as Actuator 310. Thus, sound waves analyzed and processed by Sound Converter 100 shown in FIG. 1 may be transformed by Driver 340 to a mechanical vibration energy that stimulates corresponding zones on the human hand. Additionally or alternatively, Conch 300 may draw its energy from an external power supply (not shown), a Battery 330, a combination thereof, or the like. In some exemplary embodiments, a battery, such as Battery 330, of a device, such as Conch 300, may be wirelessly charged by the docking base while in on-hook state. as illustrated in FIG. 2B

In some exemplary embodiments of the disclosed subject matter, Conch 300 may comprise an enhanced version of Driver 340, which in an addition to its previously described operations, and may be configured to perform duties associated with Sound Converter 100 shown in FIG. 1. In such embodiments, Assembler 113, a portion of I/O module 114 as shown in FIG. 1, as well as a portion of D&S 330 may become redundant.

Referring now to FIG. 3B showing several configurations of holding a device, such as Conch 333, in accordance with some exemplary embodiments of the disclosed subject matter. It should be noted that the topology of the actuators of the device may be designed to create vibrations coordinated with high, low, and intermediate audio frequencies, and to simulate the audio sense of music by means of vibrations coordinated with the tone scale from bass to soprano. The topology and the distribution of the actuators is a result of studying the hands touch sense mapping on hearing impaired participants. Test results also shows that a user (hearing impaired or not) can tactilely feel the vibrations in distributed zones according to the audio signals. After some practice, one can interpret the pattern of vibrations and distinguish if that is a guitar or piano for example.

Referring now to FIG. 4 showing an illustration of a Glove 400 comprising a plurality of Actuators 410 and a Driver 440, in accordance with some exemplary embodiments of the disclosed subject matter. It is appreciated that the human hand differs in sensitivity from other parts of the body, wherein a plurality of sensitivity zones (or pressure points) may sense delicate touch from multiple areas. For example, the hand may detect that two objects are touching the skin of the hand simultaneously in substantially close proximity zones, while other areas of the human skin have lower sensitivity. Thus, a human hand covered by Glove 400 that comprises actuators such as Actuators 410 that correspond to these zones, may provide means for differentiating between sound signals, for instance, separate musical instruments may be individually identified and then trigger the corresponding actuators. It should be noted that in some exemplary embodiments, actuators such as Actuator 410 may be connected to Driver 440 by a cable conducting the electrical signals.

Referring now to FIG. 5, showing a flowchart diagram of a method for translating sound to tactile in accordance with some exemplary embodiments of the disclosed subject matter.

In Step 510, sound signals intended for translation to tactile may be received. In some exemplary embodiments, I/O Module 114 capabilities (described above) may be utilized for obtaining sound signals from a verity of sources. Additionally, the I/O Module 114 may extract the core sound signals that represent relevant sound waves required for the translation process, from the sound signals.

In Step 520, the core sound signals may be analyzed by utilizing a processing algorithm configured to continuously determining the amplitude of each relevant frequency that comprises the core sound signal. The properties of the relevant frequencies may be predetermined and may be obtained from the memory.

In Step 530, a plurality of sound bands reflecting the sound signal content may be determined In some exemplary embodiments, the sound bands may represent the most significant components comprising the core sound. That is to say that each sound band may reflect a different acoustic effect, such as voice, music, background noise, motorcycle, singing, humming bird dog barking, a combination thereof, or the like. In some exemplary embodiments, determining the plurality of sound bands that make up the core signal may be performed by characterizing the core signal main frequencies, harmonic series, amplitude thresholds, band of adjacent frequencies, a combination thereof, or the like.

In Step 540 the plurality of sound bands may be converted to plurality electrical signals. In some exemplary embodiments, each sound band is converted to a corresponding electrical signal characterized by specific properties. The conversion may be done by employing amplitude and frequency modulation scheme adapted to represent acoustic effects (i.e. sound band) with electrical signals. Following the modulation scheme the electrical signals may be shaped for controlling actuators, in accordance to a map (e.g. look-up-table stored on the data repository) used for allocating sound bands to zones on the human skin.

In Step 550, the electrical signals may be transmitted for energizing the actuators. In some exemplary embodiments, Assembler 113 may be used to aggregate the electrical signals into at least one primary signal. Additionally or alternatively, Assembler 113 may amplifies the at least one primary signal and utilize the I/O module 114 for transmitting the at least one primary signal to the actuators by either wires or wireless communication.

In Step 560, the mechanical vibration of the actuators may be activated by matching electrical signals. In some exemplary embodiments, a driver such as D&S 126 of a device, such as Device 120, may split the at least one primary signal to its components, (i.e. the electrical signals comprising the aggregated electrical signal). In addition, the D&S 126 may amplify the electrical signals and route each one of the electrical signal to its associated actuator in order to generate applicable mechanical vibration.

In Step 570, zones on the skin that are dictated by the device may tactilely stimulate. In some exemplary embodiments, a user (hearing impaired or not) may tactilely feel the vibrations in distributed zones according to the sound bands and the amount of the actuators comprised in the device. Since a correlation between sound bands and actuators is predetermined, then correlation with skin zones may be maintained as well. Thus, the different zones may provide means for differentiating between the sounds bands, for instance, separating between musical instruments. After some practice, the user may interpret the pattern of vibrations and distinguish if that is a guitar or piano, for example.

The flowchart and block diagrams in the Figures illustrate the architecture, functionality, and operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts, or combinations of special purpose hardware and computer instructions.

The terminology used herein is for the purpose of describing particular embodiments only and is not intended to be limiting of the disclosed subject matter. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosed subject matter has been presented for purposes of illustration and description, but is not intended to be exhaustive or limited to the disclosed subject matter in the form disclosed. Many modifications and variations will be apparent to those of ordinary skill in the art without departing from the scope and spirit of the disclosed subject matter. The embodiment was chosen and described in order to best explain the principles of the disclosed subject matter and the practical application, and to enable others of ordinary skill in the art to understand the disclosed subject matter for various embodiments with various modifications as are suited to the particular use contemplated.

1. Sound translating system configured to touch human skin comprising:
a sound converter configured to process sound waves to a plurality of sound bands, wherein each sound band of the plurality of sound bands is represented by at least one electrical signal having specific electrical characteristics,
wherein said sound converter is further configured to transmit the at least one electrical signal;
at least one device comprising a plurality of actuators, wherein at least one actuator of said plurality of actuators is configured to convert the at least one electrical signal to mechanical vibration, wherein the mechanical vibration of each actuator stimulates tactile of one of a plurality of zones on the human skin, wherein the at least one actuator is dedicated to at least one predefined zone of the plurality of zones; and
wherein, said predefined zone complies with a map used to allocate sound bands to zones on the human skin.

2. The system of claim 1, wherein, the at least one actuator is further configured to modulate an amplitude and a frequency of the mechanical vibration by the specific electrical characteristics of the at least one electrical signal.

3. The system of claim 1, wherein the sound converter is selected from the group consisting of a smart phone; a notebook computer; a tablet; and a desktop computer and any combination thereof, utilized to process applications for implementing sound converter functionalities.

4. The system of claim 1, wherein the sound converter is hosted in a dedicated base unit comprising a Digital Signal Processor (DSP) programmed for implementing sound converter functionalities.

5. The system of claim 1, wherein the sound converter receives the sound waves by a microphone and wherein, the sound converter is further configured to process sound signals selected from the group consisting of Bluetooth signals; AM signals; FM signals; digital audio signals and analogue audio signals any combination thereof.

6. The system of claim 1, wherein the sound band is selected from the group consisting of a single frequency; harmonic series; a band of adjacent frequencies and any combination thereof, wherein the specific electrical characteristics of the electrical signal are indicative of at least one sound band and wherein the electrical signal is wirelessly transmitted to the at least one device.

7. (canceled)

8. The system of claim 1, wherein the device is selected from the group consisting of a bodysuit; a glove; a mat; a handball; and any combination thereof comprise a plurality of actuators, wherein each actuator of the plurality of actuators is allocated to overlap a predetermined zone on the human skin, wherein each actuator is associated with one or more sound bands selected from the group consisting of tones; acoustic harmonies; frequency ranges; and acoustic instruments and any combination thereof.

9. (canceled)

10. The system of claim 1, wherein the sound converter is further configured for assembling a plurality of the at least one electrical signal into a primary signal and transmit the primary signal to the device.

11. The system of claim 1, wherein said device further comprising a driver configured to receive a primary signal, wherein the driver is further configured to split the primary signal to a plurality of the at least one electrical signal; and wherein the at least one electrical signal is connected to at least one actuator of the plurality of actuators.

12. (canceled)

13. A sound translating device configured to touch a human skin comprising:
a sound converting component configured to process sound waves to a plurality of sound bands, wherein each sound band of the plurality of sound bands is represented by at least one electrical signal having specific electrical characteristics,
a plurality of actuators, wherein at least one actuator of said plurality of actuators is configured to convert the at least one electrical signal to mechanical vibration, wherein the mechanical vibration of each actuator stimulates tactile of one of a plurality of zones on the human skin, wherein the at least one actuator is dedicated to at least one predefined zone of the plurality of zones; and
wherein, said predefined zone complies with a map used to allocate sound bands to zones on the human skin.

14. The system of claim 13, wherein, the at least one actuator is further configured to modulate an amplitude and a frequency of the mechanical vibration by the specific electrical characteristics of the at least one electrical signal.

15. The system of claim 13, wherein the sound converting component utilizes a Digital Signal Processor (DSP) programmed for implementing sound converting functionalities.

16. The device of claim 13, wherein the sound converting component receives the sound waves by a microphone and wherein, the sound converting component is further configured to process sound signals selected from the group consisting of Bluetooth signals; AM signals; FM signals; digital audio signals; analogue audio signals and any combination thereof.

17. The device of claim 13, wherein the sound band is selected from the group consisting of a single frequency; harmonic series; a band of adjacent frequencies; and any combination thereof and the specific electrical characteristics of the electrical signal are indicative of at least one sound band.

18. (canceled)

19. The device of claim 13, wherein the device is selected from the group consisting of a bodysuit; a glove; a mat a handball and any combination thereof comprise a plurality of actuators, wherein each actuator of the plurality of actuators is allocated to overlap a predetermined zone on the human skin and, wherein each actuator is associated with one or more sound bands selected from the group consisting of tones; acoustic harmonies; frequency ranges; and acoustic instruments; and any combination thereof.

20. (canceled)

21. The system of claim 13, wherein the sound converter component is further configured for assembling a plurality of the at least one electrical signal into a primary signal and transmit the primary signal to the device.

22. The system of claim 13, wherein said device further comprising a driver configured to receive a primary, wherein the driver is further configured to disband the primary signal to a plurality of the at least one electrical signal; and wherein the at least one electrical signal is connected to at least one actuator of the plurality of actuators.

23. The system of claim 13, wherein said device and said driver are physically separated.

24. A method for translating sound into mechanical vibration on human skin, the method comprising:
receiving sound signals selected from the group consisting of microphone signals; Bluetooth signals; AM signals; FM signals; digital audio signals; analogue audio signals; and any combination thereof.
continuously analyzing the sound signals amplitude and frequencies;
continuously determining a plurality of sound bands to reflect content of the sound based on the sound signals amplitude and frequencies;
continuously converting each sound band of the plurality of sound bands to an electrical signal of a plurality of electrical signals, wherein each electrical signal of the plurality of electrical signals have a specific electrical characteristics that represent the sound band;
transmitting the plurality of electrical signals to a device comprising a plurality of actuators, wherein each actuator of the plurality of actuators is activated by a matching electrical signal of the plurality of electrical signals;
wherein each actuator transforms the energy of the matching electrical signal to a mechanical vibration; and
wherein the mechanical vibration stimulates tactile of one of a plurality of zones on the human skin

25. The method of claim 24, wherein said converting further comprises modulating an amplitude and a frequency of an electrical signal to satisfy mechanical vibration characteristics of an actuator.
